(19) United States
(12) Patent Application Publication
(10) Pub. No.: US 2026/0287434 A1
(43) Pub. Date: Sep. 24, 2026

(54) SYSTEMS AND METHODS FOR QUANTIFYING LIGHT INTENSITY

(71) Applicant: INTUITIVE SURGICAL OPERATIONS, INC., Sunnyvale, CA (US)

(72) Inventors: John A Barton, San Jose, CA (US); Gerald M. Castro, Sunnyvale, CA (US); Mathew D. Clopp, Santa Clara, CA (US); Ryan K. Gong, San Jose, CA (US); Jonathan D. Halderman, Sunnyvale, CA (US); Arundhati N. Kabe, Cupertino, CA (US); Kyelo C. Torres, San Jose, CA (US); Stefan Eberle, Mountain View, CA (US)

(21) Appl. No.: 19/484,662

(22) PCT Filed: May 15, 2024
(86) PCT No.: PCT/US2024/029395

§ 371 Date: Nov. 14, 2025

Related U.S. Application Data
(60) Provisional application No. 63/502,544, filed on May 16, 2023.

(51) Int. Cl. G01J 5/10; G01J 5/0806; G01J 5/0821

(52) U.S. Cl. CPC G01J 5/10; G01J 5/0806; G01J 5/0821

(57) ABSTRACT

A system may comprise a light sensor, a light source configured to generate a light beam, and a light probe. The light probe may include a first end portion configured to extend into the light beam to receive a light sample from the light beam and may include a second end portion coupled to the light sensor to deliver the light sample to the light sensor. The first end portion of the light probe may be configured to divert a reflected light, from a reflective member, away from the light sensor.

### CROSS-REFERENCED APPLICATIONS

This application claims priority to and benefit of U.S. Provisional Application No. 63/502,544 filed May 16, 2023 and entitled “Systems and Methods for Quantifying Light Intensity,” which is incorporated by reference herein in its entirety.

### FIELD

Examples described herein relate to systems and methods for quantifying light intensity. More particularly, examples may relate to quantifying light intensity in a forward-transmission system with back reflection.

### BACKGROUND

Minimally invasive medical techniques may generally be intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions an operator may insert minimally invasive medical instruments such as therapeutic instruments, diagnostic instruments, imaging instruments, and surgical instruments. Some minimally invasive medical instruments may include illumination systems. Systems and methods are needed to accurately measure light intensity to, for example, provide a closed loop optical power system.

### SUMMARY

The following presents a simplified summary of various examples described herein and is not intended to identify key or critical elements or to delineate the scope of the claims.

In some examples, a system may comprise a light sensor, a light source configured to generate a light beam, and a light probe. The light probe may include a first end portion configured to extend into the light beam to receive a light sample from the light beam and may include a second end portion coupled to the light sensor to deliver the light sample to the light sensor. The first end portion of the light probe may be configured to divert a reflected light, from a reflective member, away from the light sensor.

In some examples, a system may comprise a light source configured to generate a light beam, a light sensor, and a light probe. The light probe may include a first end portion configured to extend into the light beam to receive a light sample from the light beam and may include a second end portion coupled to the light sensor to deliver the light sample to the light sensor. The first end portion of the light probe includes an acceptance region angled to receive the light sample.

In some example, a system may comprise a light source configured to generate a light beam, a light sensor, and a light probe. The light probe may include a first end portion configured to extend into the light beam to receive a light sample from the light beam and may include a second end portion coupled to the light sensor to deliver the light sample to the light sensor. The light probe may be configured to accept a first light portion of the light beam from a first direction into the light probe and to divert a second light portion of the light beam from a second direction away from the light probe.

In some examples, a method may comprise receiving a sampled light at a distal surface of a light probe from an acceptance region of the light probe and directing the sampled light from the distal surface of the light probe toward a photodetector. The method may also comprise receiving a reflected light at the distal surface of the light probe outside of the acceptance region of the light probe, directing the reflected light away from the photodetector, and analyzing an intensity of the sampled light.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a schematic diagram of an illumination system, according to some examples.

FIG. 2 is a schematic diagram of a photodetector, according to some examples.

FIG. 3 is a detailed view of a portion of the illumination system of FIG. 1.

FIG. 4A is a schematic diagram of an illumination system, according to some examples.

FIG. 4B is a schematic diagram of an illumination system, according to some examples.

FIG. 4C is a schematic diagram of an illumination system, according to some examples.

FIG. 5 is a schematic diagram of an illumination system, according to some examples.

FIG. 6 is a schematic diagram of an illumination system, according to some examples.

FIG. 7A illustrates a vision system, according to some examples.

FIG. 7B illustrates a distal end of an endoscopic instrument system, according to some examples.

FIG. 8 is a flow chart illustrating a method for controlling a light source, according to some examples.

FIG. 9 is a robotically-assisted medical system, according to some examples.

Examples of the present disclosure and their advantages are best understood by referring to the detailed description that follows. It should be appreciated that like reference numerals are used to identify like elements illustrated in one or more of the figures, wherein showings therein are for purposes of illustrating examples of the present disclosure and not for purposes of limiting the same.

### DETAILED DESCRIPTION

The technology described herein provides techniques and treatment systems for quantifying light intensity and may be used, for example, in an endoscopic imaging system to provide an accurate measurement of light intensity from a light source, while minimizing the detection of reflected light within the system. The described technology may be used in performing procedures though artificially created lumens or any endoluminal passageway or cavity, including in a patient trachea, colon, intestines, stomach, liver, kidneys and kidney calices, brain, heart, respiratory system, circulatory system including vasculature, fistulas, and/or the like.

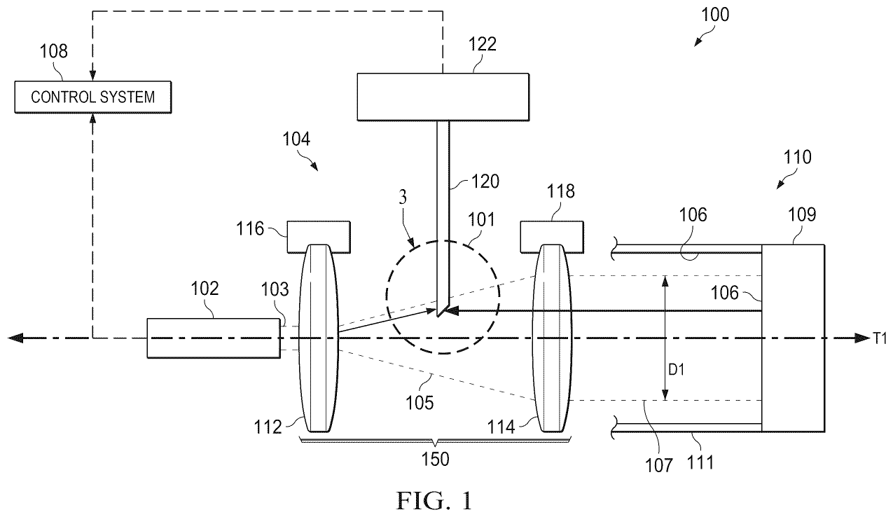

FIG. 1 provides a schematic diagram of an illumination system 100. The illumination system 100 may, for example, provide illumination for a surgical scene within a patient anatomy and a control system to control the amount of light delivered to the surgical scene. The illumination system may include a light source 102 and a light sensor system 104. The light source 102 may provide input light beam 103 to the illumination system. The illumination system 100 may also include a reflective member 106 which may include any surface that reflects light from the light source 102. In some examples, the reflective member 106 may be fixed relative to the light source 102 and the light sensor system 104. In other examples, the reflective member 106 may be detachable from or movable relative to the light source 102 and light sensor system 104. In some examples, the illumination system may include a control system 108 that receives the sampled light intensity information from the light sensor system 104 to provide a control loop for controlling power to the light source 102 to reach a target optical output power. The light sensor system 104 may reject light reflected from the reflective member 106 back into the illumination system 100 to provide a more accurate measure of the input light beam 103. In some examples, the control system 108 may be part of a control system of an endoscopic instrument system or a robot-assisted medical system (e.g., control system 712).

The light source 102 may generate visible light (e.g., white light or components thereof) and/or non-visible light. Non-visible light may be in the infrared spectrum with wavelengths between approximately 700 nm and 1 mm. Non-visible light may be in the ultraviolet spectrum with wavelengths between approximately 10 nm and 400 nm. The light may be generated, for example, from light emitting devices (LED's), lasers, halogen bulbs, xenon bulbs, and/or metal halide bulbs. In some examples, the light source 102 may have four or more light channels (e.g., red light, green light, blue light, and near infrared light (NIR)). In some examples, the light sensor system 104 may be effective for use with smaller beam light sources such as laser light sources.

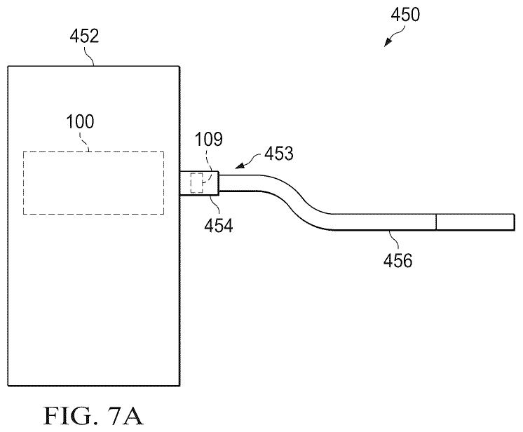

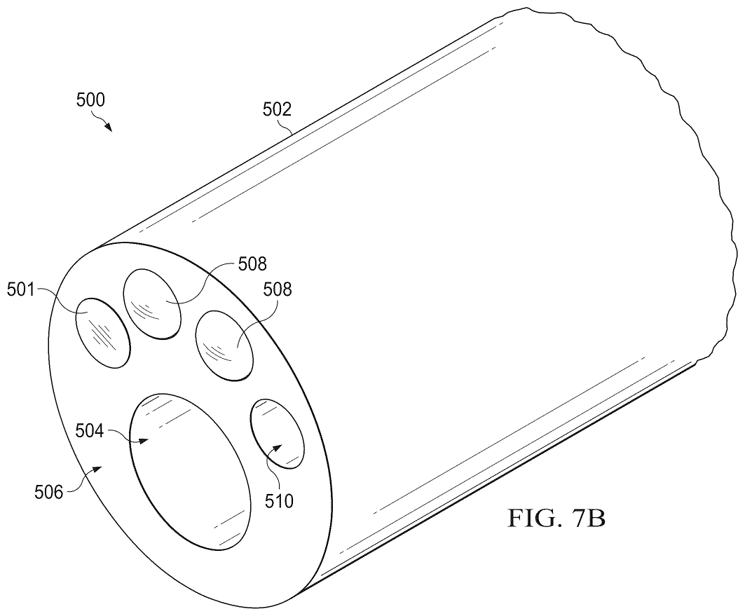

The reflective member 106 may include, for example, a surface of a component 109 located at or near a distal end portion 110 of the illumination system. In some examples (see, e.g. FIG. 7B), the component 109 may be a cover component located at a proximal or distal tip of an endoscopic instrument system inserted into a patient anatomy. The cover component may be a transparent protective element to shield optical fibers and/or lenses of the illumination system from bodily fluids or contact with tissue. In some examples (see, e.g., FIG. 7A), the component 109 may be an interface component at an interface where an endoscopic instrument system couples to vision assembly 452. In such an example, the illumination system 100 may be housed within the vision assembly 452, and the interface component may be part of the vision assembly 452, part of a detachable coupling member, or in a proximal end 453 of the endoscopic instrument system. The component 109 may be formed, for example, from crystalline material such as a natural sapphire gemstone, crystallized aluminum oxide, glass, plastic, acrylic, or another type of generally transparent material that may transmit some or most impinging light but may also reflect at least a portion of the impinging light received from some directions. In some examples, the reflective member 106 may be a reflective surface of a light housing 111 (e.g., a vision assembly housing or an endoscopic instrument system housing), a fixture, a clamping element, or any other structural component of the illumination system 100 or an endoscopic instrument system coupled to or housing the illumination system. In these examples, the reflective surface may be formed of glass, metal, plastic, or other partially reflective materials.

Optionally, the illumination system 100 may include an expanding lens system 112. The one or more lenses of the expanding lens system 112 may expand the input light beam 103 emitted from the light source 102 to generate an expanded light beam 105. Optionally, the illumination system 100 may include a collimating lens system 114. The one or more lenses of the collimating lens system 114 may generate a collimated light beam 107 following expansion by the expanding lens system 112. In some examples a diameter D1 of the collimated light beam may be approximately 20 mm. In other examples, the beam diameter may be smaller or larger. Lens holders 116, 118 may fix the lens systems 112, 114, respectively, within the housing 111 or other framework of the illumination system 100.

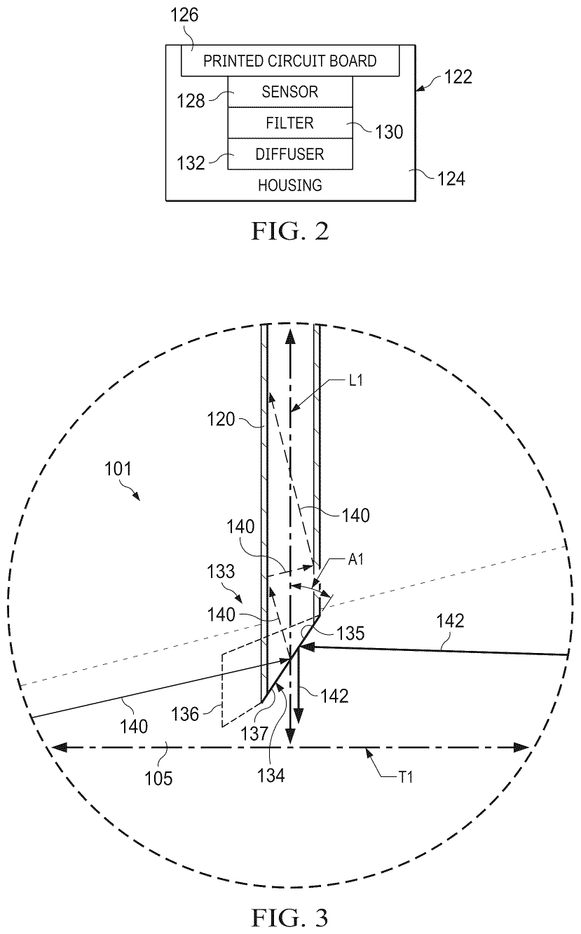

The light sensor system 104 may include a light probe 120 having a generally flat surface coupled to, in abutment with, or in close proximity to a photodetector 122. FIG. 2 provides a schematic diagram of the photodetector 122. The photodetector 122 (e.g., a photodiode) may include a housing 124 containing a printed circuit board 126. The printed circuit board 126 may be coupled to a sensor 128, an optional filter 130, and an optional diffuser 132. The sensor 128 may include a light sensing computer chip configured to sense light intensity for one or more wavelengths of light. In some examples, different sensors or different light sensor systems may be used to sense different ranges of light. For example, with a four channel light source (e.g., red, green, blue, NIR channel), four separate sensors or four separate light sensor systems may be used to measure the light intensity for each channel. The filter 130 may be, for example, a neutral density filter for reducing light intensity to help control the brightness. The diffuser 132 may smooth the signal, prevent issues with exact alignment of the fiber to the sensor, and make it more tolerant to minor shifts. The photodetector 122 may be used to measure the optical power of the detected light by converting detected photons into a measurable electrical current.

FIG. 3 provides a detailed view of a region 101 of the illumination system 100, including a distal end portion 133 of the light probe 120 extended into the light beam 105 to obtain a light sample. The light probe 120 may include, for example, a single optical fiber, a solid glass rod, or an optical fiber bundle. The light probe 120 may be formed of material (e.g., glass) that may sustain high temperatures from laser light sources. For example, the light probe 120 may operate within temperatures ranging from −190 C to +390 C. In some examples, the light probe 120 may be formed from an optical fiber bundle held within a stainless steel hypotube by an adhesive, with the whole assembly may be polished at a distal tip. In some examples, the light probe 120 maybe coated with a reflective material such as aluminum or silver. In some alternative examples, instead of an optical fiber or glass rod, a probe may include a beam splitter with mirror system including a series of mirrors to direct light toward the photodetector 122.

A distal end portion 133 of the light probe 120 may have a polished surface 134 polished at an angle A1 to form a light needle. The angle A1 may be the angle between the polished surface 134 and the central longitudinal axis L1 of the light probe 120. The angle A1 may produce a light acceptance region 136 which may be, for example, a generally cone-shaped acceptance region to receive the light sample. In some examples, the angled surface 134 formed by the angle A1 may be, for example, between 30 and 35 degrees to create a light acceptance region 136 that reliably captures sufficient light to evaluate the intensity of the light from the light source 102. In other examples, suitable angles A1 may be between 20 and 45 degrees. Probe tips may be prone to breakage if the angle A1 becomes too small. The direction, rotation, or orientation of the light acceptance region 136 may be selected to maximize capture of light from the direction of the light source and minimize capture of light from reflected surfaces distal of the probe. The size and direction of the light acceptance region 136 and thus the performance and characteristics of the light probe 120 may be determined, at least in part, by the angle A1 and the numerical aperture of the optical fiber(s) or rod forming the probe. The numerical aperture may be a function of the refractive index of the fiber core and the refractive index of the fiber cladding.

The light acceptance region 136 may be angled or directed receive the light sample. In some examples, the light acceptance region 136 may broaden, such as in a cone shape, from the polished surface 134 toward the light source 102 or otherwise in a direction from which the expanded light beam 105 emanates. Sampled light received at a first side 135 of the surface 134 through the light acceptance region 136 may be gathered into the light probe 120, and light received at a second side 137 of the surface 134 from directions not within the light acceptance region 136 (e.g., light reflected from reflective members 106) may be rejected from the light probe 120. The polished distal tip of light probe 120, including the surface 134, may serve as a prism to direct light from the acceptance region 136 toward the photodetector 122 at the opposite end of the light probe 120. As an example, light 140, which may be a portion of the expanded light beam 105, may be transmitted into the light acceptance region 136 of the light probe 120, may impinge upon the surface 134, and may be transmitted by total internal reflection within the light probe 120 toward the photodetector 122. Light 142 reflected from any of various reflective members 106, including surfaces of the component 109 and/or the housing 111, may strike the surface 134 of the light probe 120 outside of the light acceptance region 136. Thus, the light 142 may be diverted away from the light probe 120 and not propagate toward the photodetector 122. The diversion of the light 142 may include any rejection or redirection of a light beam whether by reflection, refraction, diffraction, or absorption. Consequently, the light received by the photodetector 122 for analysis and closed loop control of the light source 102 may be light that is entirely or primarily from the light source 102, with minimal or no reflected or stray light from other surfaces of the illumination system 100.

The light probe 120 may be held in a fixed position and orientation with respect to the light source 102. In some examples, as shown in FIG. 1, the distal end portion 133 of the light probe 120 may be inserted into the light beam in a region 150 between the expanding lens system 112 and the collimating lens system 114. In some examples the longitudinal distance of the region 150 between the lens systems may be, for example, approximately 22 mm with an approximate diameter of 2.5 mm. In this example, the central longitudinal axis L1 of the light probe 120 may be oriented generally perpendicular to a transmission axis T1 of the light source.

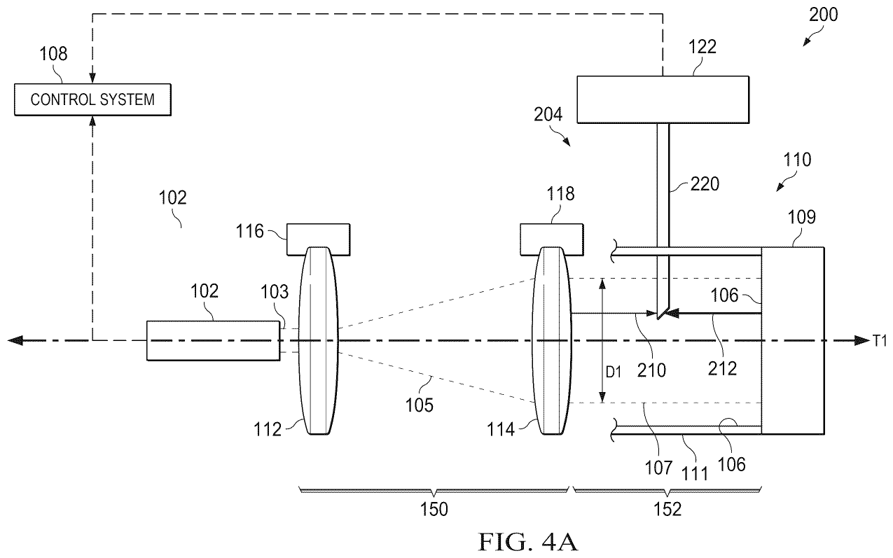

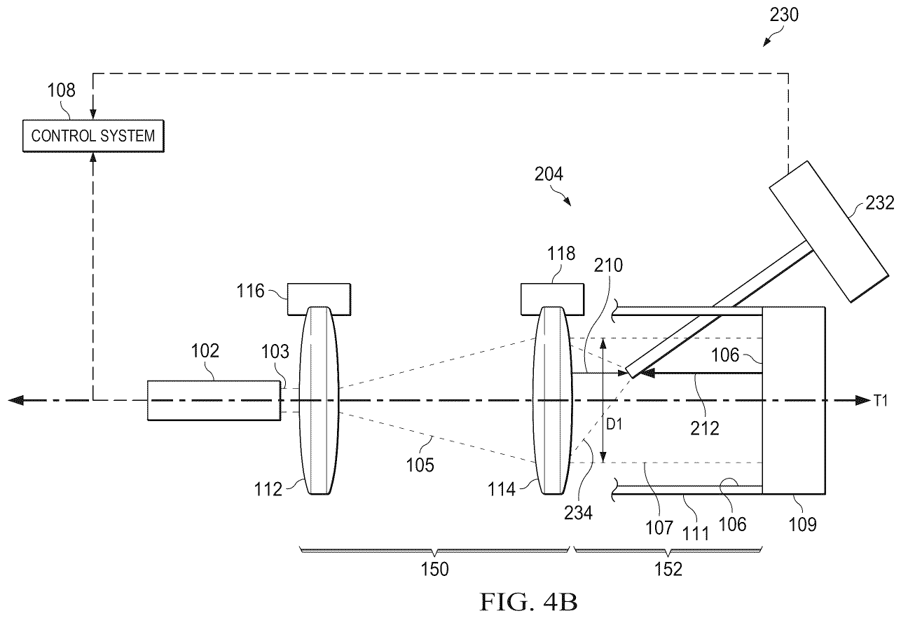

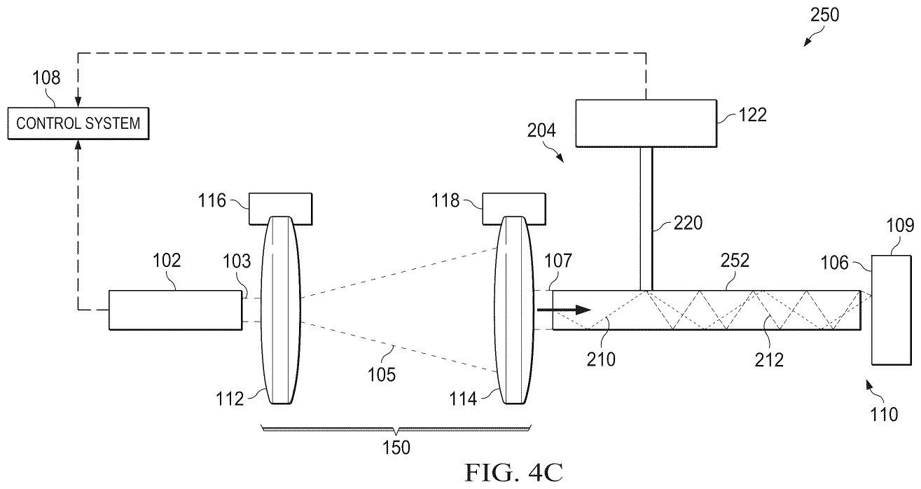

In an alternative example, as shown in FIG. 4A, an illumination system 200 may include the same or similar components as the illumination system 100, with differences as described. In this example, a light sensor system 204 (e.g., similar to the light sensor system 104 with differences as described) may probe the collimated light beam in a region 152, distal of the collimating lens system 114. The acceptance region of a light probe 220 of the light sensor system 204 may be modified (as compared to the light sensor system 104) by adjusting the numerical aperture and/or polished angle of the probe to primarily or only accept light 210 transmitted from the light source and primarily or entirely reject light 212 reflected from the component 109 or other reflective members 106. In other examples, the light probe may extend into the light beam between the light source and the lens system.

In an alternative example, as shown in FIG. 4B, an illumination system 230 may include the same or similar components as the illumination system 100, with differences as described. In this example, a light probe 232 may probe the light in a collimated region or uncollimated region. The light probe 232 may be polished and/or cleaved flat (e.g., normal to the central longitudinal axis of the light probe) and the whole probe may be tilted to create a desired light acceptance region 234 towards the source light beam.

In an alternative example, as shown in FIG. 4C, an illumination system 250 may include the same or similar components as the illumination system 100, with differences as described. In this example, light may be sensed at an optical homogenizing element. In this example, an optical homogenizing element 252 (e.g., a hexagonal light rod), at or near an output of a lens system comprising one or more lenses, may carry the source light 210 and the reflected light 212. The light probe 220 placed at, on, or adjacent to the homogenizing element 252 and may sense the light from the element. In this example, instead of diverting the reflected beam 212, sampling occurs in such a high forward intensity location that the signal to noise ratio is high enough that the reflection may be negligible in the reading. In this or other examples, an index matching element including as index matching material (e.g., silicone) may be incorporated into the tip of the light probe or located between the light probe and the element 252.

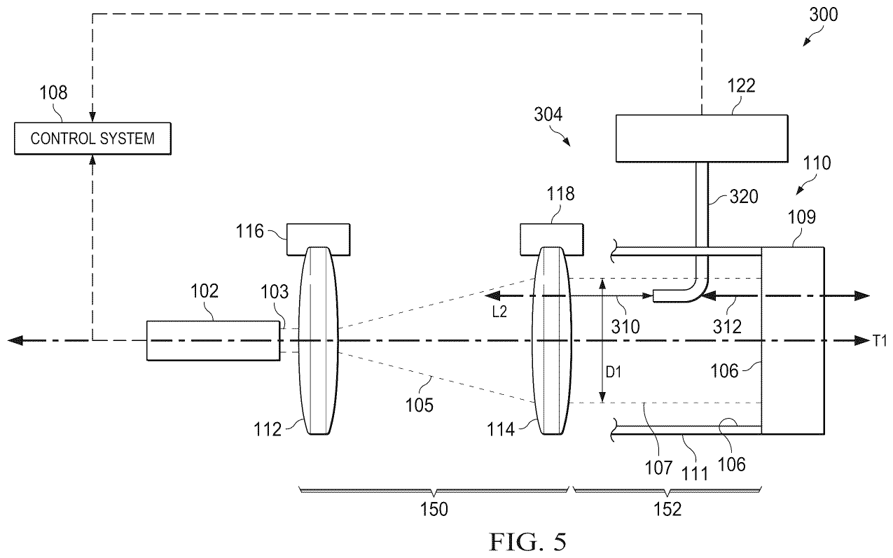

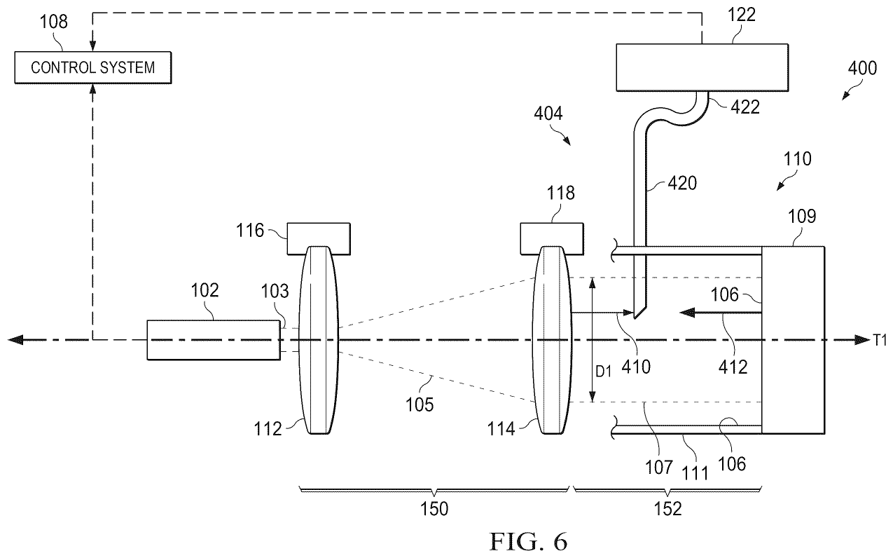

In an alternative example, as shown in FIG. 5, an illumination system 300 may include the same or similar components as the illumination system 100, with differences as described. In this example, a light sensor system 304 (e.g., similar to the light sensor system 104 with differences as described) may probe the collimated light beam in the region 152, distal of the collimating lens system 114. In this example, a central longitudinal axis L2 of a distal end portion of a light probe 320 of the light sensor system 304 may be substantially parallel to the direction of the collimated light and/or the transmission axis T1 of the light source. The acceptance region of the light probe 320 may be modified (as compared to the light sensor system 104) by adjusting the numerical aperture and/or polished angle of the probe to primarily or only accept light 310 transmitted from the light source and primarily or entirely reject light 312 reflected from the component 109 or other reflective members 106. The polished surface of the distal tip of the light probe 320 may be, for example, generally perpendicular to the axis L2. In other alternative examples, the probe may intersect the light beam at a variety of angles, and the acceptance regions may be adjusted accordingly to receive light from the light source and reject reflected light from the cover or other surfaces.

In an alternative example, as shown in FIG. 6, an illumination system 400 may include the same or similar components as the illumination system 100, with differences as described. In this example, a light sensor system 404 (e.g., similar to the light sensor system 104 with differences as described) may probe the collimated light beam in the region 152, distal of the collimating lens system 114. In this example, a light probe 420 of the light sensor system 404 may be coupled to the photodetector 122 by a fiber optic cable or a length of optical fiber 422. The acceptance region of a light probe 420 of the light sensor system 404 may be modified (as compared to the light sensor system 104) by adjusting the numerical aperture and/or polished angle of the probe to primarily or only accept light 410 transmitted from the light source and primarily or entirely reject light 412 reflected from the component 109 or other reflective members 106.

FIG. 7A illustrates a vision system 450 including a vision assembly 452 that houses the illumination system (or the illuminations systems 200, 300, 400). The vision assembly 452 may, optionally, include one or more display screens, power generation components, image processing components, and/or information systems. In some examples, the vision assembly may be a mobile vision cart. The vision system 450 may further include a coupling member 454 and an endoscopic instrument system 456 detachably coupled to the to the vision assembly 452 by the coupling member. In this example, the component 109 may be an interface component located within the coupling member 454. In other examples, the component 109 may be an interface component located with the vision assembly 452 or at a proximal end 458 of the endoscopic instrument system 456.

FIG. 7B illustrates a distal end portion of an endoscopic instrument system 500. The endoscopic instrument system 500 may include an illumination system (e.g., illumination system 100, 200, 300, 400). The endoscopic instrument system 500 may include a rigid or flexible elongate body 502. In some examples, the illumination system housing 111 may be the body 502. A cover component 501 of the illumination system may be disposed at a distal end 506 of the elongate body. The cover component may be a component 109 with a surface that is a reflective member 106. A working channel 504 may extend through the elongate body 502 to the distal end 506 of the body to provide passage for removable instrument systems and allow instruments to be exchanged during a procedure. The working channel may also or alternatively allow fluid passage or otherwise provide access between proximal and distal portions of the elongate flexible instrument. The endoscopic instrument system 500 may also include an imaging system 508, such as a stereoscopic camera, and an irrigation system 510.

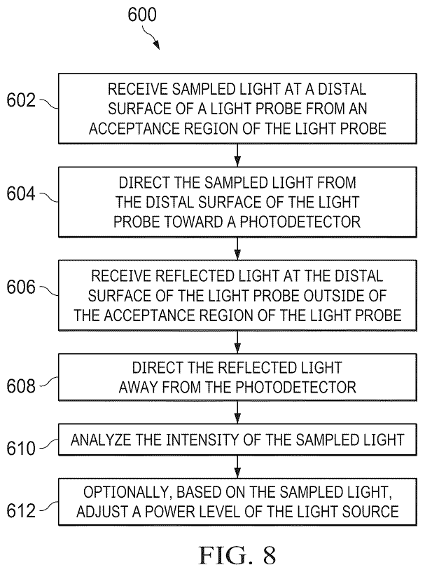

FIG. 8 is a flowchart illustrating a method 600 for controlling a light source by detecting light transmitted from the light source while minimizing the detection of light from reflected surfaces such as a distal cover. The detected light may be used to determine the intensity of light from the light source and may be used to control power to brighten or dim the light source. The method 600 is illustrated as a set of operations or processes that may be performed in the same or in a different order than the order shown in FIG. 8. One or more of the illustrated processes may be omitted in some examples of the method. Additionally, one or more processes that are not expressly illustrated in FIG. 8 may be included before, after, in between, or as part of the illustrated processes. In some examples, one or more of the processes of method 800 may be implemented, at least in part, by a control system executing code stored on non-transitory, tangible, machine-readable media that when run by one or more processors (e.g., the processors of a control system) may cause the one or more processors to perform one or more of the processes.

At a process 602, a light sample may be received at a distal surface of a light probe. The sampled light may be light that falls within an acceptance region of the light probe. The acceptance region may be generally oriented a direction to receive light from a light source. The light probe (e.g., a light probe 120) may be a component of a light sensor system (e.g., light sensor system 104) of an illumination system (e.g., the illumination system 100) that includes the light source (e.g., light source 102). The light probe may be inserted into a light beam at a position immediately distal of the light source, at a location distal of an expansion lens system, or at a location distal of a collimating lens system, for example.

At a process 604, the sampled light may be directed through the distal surface of the light probe toward a photodetector. For example, sampled light 140 may enter the acceptance region 136 of the light probe 120 and may be reflected off the surface 134 and into the probe. The sampled light 140 may be directed by total internal reflectance along the length of the light probe 120 toward the photodetector 122.

At a process 606, reflected light may be received at the distal surface of the light probe outside of the acceptance region of the light probe. For example, the light 142 may be diverted from a reflective surface 106 such as surface of a housing 111 or a component 109. The light 142 may be received at the surface 134 of the light probe 120 from a direction outside of the light acceptance region 136. The diversion of the light 142 may include any rejection or redirection of a light beam whether by reflection, refraction, diffraction, or absorption.

At a process 608, the reflected light may be directed away from the photodetector. For example, the light 142 may be directed away from the light probe 120 and thus away from the photodetector 122. Consequently, the light that reaches the photodetector 122 for analysis is comprised only or primarily of light directly from the light source 102 and has little or no contribution from stray or reflected light that approaches the probe from outside the acceptance region.

At a process 610, the sampled light may be analyzed to determine an intensity of the sampled light. For example, the photodetector 122 may determine light intensity (e.g., optical power) from the sampled light 140. Because the reflected light 142 is omitted from the sample, the determined light intensity may omit any contribution caused by reflected or stray light within the illumination system housing and thus may provide a more accurate indicator of the light intensity of the light source 102.

At an optional process 612, the light source may be adjusted based on the determined intensity of the sampled light. For example, the measured intensity of the sampled light 140 may be compared to a target light intensity or optical power for the light source 102, and power to the light source may be adjusted to create more or less light output from the light source.

In some examples, the light sensor system (e.g., light sensor system 104) may have a sensor output measurement (e.g., signal/(signal+noise). As compared to traditional ambient light measurement techniques that include the measurement of stray and reflected light, light sampled with the disclosed light probes may provide a more accurate measurement of the light delivered by the illumination system. In some examples, with a near infrared light source and a crystalline cover, a traditional ambient light sensor may register approximately 38% more light due to the reflected light from the cover. With a light probe according to any of the examples provided herein, the light sensor may register less than 3% more light due to the reflected light from the cover. Thus, the examples using the light probes and the techniques for use provided herein may allow for more accurate measurement of the light intensity and, accordingly, more accurate closed-loop control of the light source.

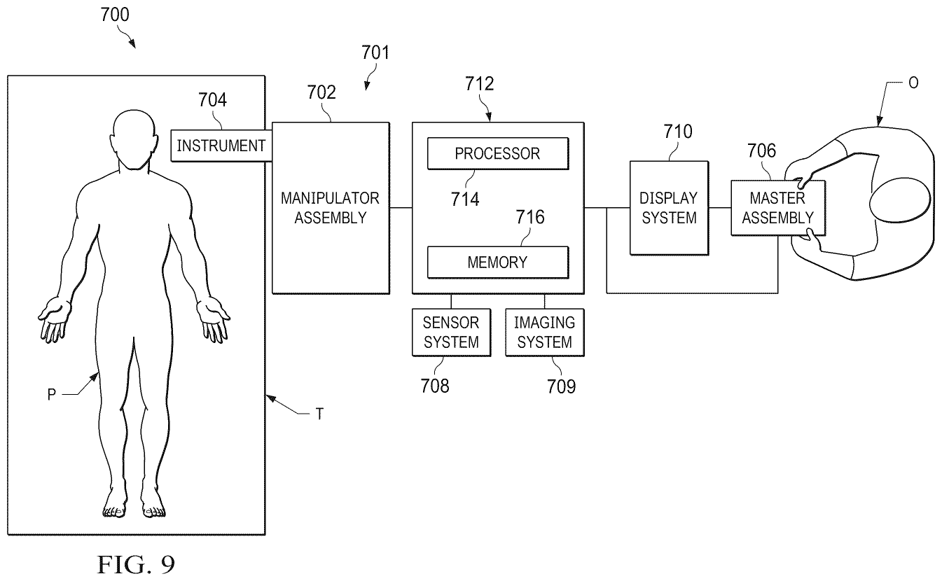

In some examples, the illumination systems and/or endoscopic instrument systems used herein may be components of a robotically-assisted medical system. FIG. 9 illustrates a robotically-assisted medical system, according to some examples. As shown in FIG. 9, a robotically-assisted medical system 700 may include a manipulator assembly 702 for operating a medical instrument 704 (e.g., illumination system 100, an endoscopic instrument system 500, or any of the systems or instrument components described herein) in performing various procedures on a patient P positioned on a table T in a surgical environment 701. The manipulator assembly 702 may be teleoperated, non-teleoperated, or a hybrid teleoperated and non-teleoperated assembly with select degrees of freedom of motion that may be motorized and/or teleoperated and select degrees of freedom of motion that may be non-motorized and/or non-teleoperated. A master assembly 706, which may be inside or outside of the surgical environment 701, generally includes one or more control devices for controlling manipulator assembly 702. The control devices may include any number of a variety of input devices, such as joysticks, trackballs, data gloves, trigger-guns, hand-operated controllers, voice recognition devices, body motion or presence sensors, and/or the like. To provide the operator O a strong sense of directly controlling instruments 704 the control devices may be provided with the same degrees of freedom as the associated medical instrument 704. In this manner, the control devices provide the operator O with telepresence or the perception that the control devices are integral with medical instruments 704.

Manipulator assembly 702 supports medical instrument 704 and may optionally include a kinematic structure of one or more non-servo controlled links and/or one or more servo controlled links. The manipulator assembly 702 may optionally include a plurality of actuators or motors that drive inputs on medical instrument 704 in response to commands from a control system 712. The actuators may optionally include transmission or drive systems that when coupled to medical instrument 704 may advance medical instrument 704 into a naturally or surgically created anatomic orifice. Other transmission or drive systems may move the distal end of medical instrument in multiple degrees of freedom, which may include three degrees of linear motion (e.g., linear motion along the X, Y, Z Cartesian axes) and in three degrees of rotational motion (e.g., rotation about the X, Y, Z Cartesian axes). The manipulator assembly 702 may support various other systems for irrigation, treatment, or other purposes. Such systems may include fluid systems (including, for example, reservoirs, heating/cooling elements, pumps, and valves), generators, lasers, interrogators, and ablation components.

Robotically-assisted medical system 700 also includes a display system 710 for displaying an image or representation of the surgical site and medical instrument 704 generated by an imaging system 709 which may include an endoscopic instrument system. Display system 710 and master assembly 706 may be oriented so an operator O can control medical instrument 704 and master assembly 706 with the perception of telepresence. Any of the previously described graphical user interfaces may be displayable on a display system 710 and/or a display system of an independent planning workstation.

In some examples, the endoscopic instrument system components of the imaging system 709 may be integrally or removably coupled to medical instrument. However, in some examples, a separate endoscope, attached to a separate manipulator assembly may be used with medical instrument 704 to image the surgical site. The imaging system 709 may be implemented as hardware, firmware, software, or a combination thereof which interact with or are otherwise executed by one or more computer processors, which may include the processors of the control system 712.

A sensor system 708 may include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument 704. The sensor system 708 may also include temperature, pressure, force, or contact sensors or the like.

Robotically-assisted medical system 700 may also include control system 712. Control system 712 includes at least one memory 716 and at least one computer processor 714 for effecting control between medical instrument 704, master assembly 706, sensor system 708, and display system 710. Control system 712 also includes programmed instructions (e.g., a non-transitory machine-readable medium storing the instructions) to implement instrument actuation using the robotically-assisted medical system including for navigation and steering.

Control system 712 may optionally further include a virtual visualization system to provide navigation assistance to operator O when controlling medical instrument 704 during an image-guided surgical procedure. Virtual navigation using the virtual visualization system may be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 712 may use a pre-operative image to locate the target tissue (using vision imaging techniques and/or by receiving user input) and create a pre-operative plan.

In the description, specific details have been set forth describing some examples. Numerous specific details are set forth to provide a thorough understanding of the examples. It will be apparent, however, to one skilled in the art that some examples may be practiced without some or all these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions. Not all the illustrated processes may be performed in all examples of the disclosed methods. Additionally, one or more processes that are not expressly illustrated in may be included before, after, in between, or as part of the illustrated processes. In some examples, one or more of the processes may be performed by a control system or may be implemented, at least in part, in the form of executable code stored on non-transitory, tangible, machine-readable media that when run by one or more processors may cause the one or more processors to perform one or more of the processes.

Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one example may be combined with the features, components, and/or steps described with respect to other examples of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

One or more elements in examples of this disclosure may be implemented in software to execute on a processor of a computer system such as control processing system. When implemented in software, the elements of the examples of this disclosure may be code segments to perform various tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and/or magnetic medium. Processor readable storage device examples include an electronic circuit; a semiconductor device, a semiconductor memory device, a read only memory (ROM), a flash memory, an erasable programmable read only memory (EPROM); a floppy diskette, a CD-ROM, an optical disk, a hard disk, or other storage device. The code segments may be downloaded via computer networks such as the Internet, Intranet, etc. Any of a wide variety of centralized or distributed data processing architectures may be employed. Programmed instructions may be implemented as a number of separate programs or subroutines, or they may be integrated into a number of other aspects of the systems described herein. In some examples, the control system may support wireless communication protocols such as Bluetooth, Infrared Data Association (IrDA), HomeRF, IEEE 802.11, Digital Enhanced Cordless Telecommunications (DECT), ultra-wideband (UWB), ZigBee, and Wireless Telemetry.

The systems and methods described herein may be suited for imaging and treatment , via natural or surgically created connected passageways, in any of a variety of anatomic systems, including the lung, colon, the intestines, the stomach, the liver, the kidneys and kidney calices, the brain, the heart, the circulatory system including vasculature, and/or the like. While some examples are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the embodiments of the invention are not described with reference to any particular programming language. It will be appreciated that a variety of programming languages may be used to implement the teachings of the invention as described herein.

In some instances well known methods, procedures, components, and circuits have not been described in detail so as not to unnecessarily obscure aspects of the examples. This disclosure describes various instruments, portions of instruments, and anatomic structures in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., in one or more degrees of rotational freedom such as roll, pitch, and/or yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, or orientations measured along an object.

While certain illustrative examples of the invention have been described and shown in the accompanying drawings, it is to be understood that such examples are merely illustrative of and not restrictive on the broad invention, and that the examples of the invention are not limited to the specific constructions and arrangements shown and described, since various other modifications may occur to those ordinarily skilled in the art.

1. A system comprising:
a light source configured to generate a light beam;
a light sensor; and
a light probe including a first end portion configured to extend into the light beam to receive a light sample from the light beam and including a second end portion coupled to the light sensor to deliver the light sample to the light sensor,
wherein the first end portion of the light probe is configured to divert a reflected light, from a reflective member, away from the light sensor.

2. The system of claim 1, wherein the light source includes a laser or a light emitting diode.

3. (canceled)

4. The system of claim 1, wherein the light source generates a light in an infrared spectrum.

5. The system of claim 1, further comprising a photodetector including the light sensor.

6-8. (canceled)

9. The system of claim 1, wherein the light probe is configured to extend into the light beam between the light source and the reflective member.

10. The system of claim 1, further comprising a lens system.

11. The system of claim 10, wherein the lens system includes an expanding lens to expand the light beam.

12. The system of claim 11, wherein the lens system includes a collimating lens to collimate the light beam.

13. The system of claim 12, wherein the light probe is configured to extend into the light beam between the expanding lens and the collimating lens.

14. The system of claim 12, wherein the light probe is configured to extend into the light beam between the collimating lens and the reflective member.

15. The system of claim 1, wherein the first end portion of the light probe includes a surface to divert the reflected light, the surface angled relative to a central longitudinal axis of the light probe.

16. The system of claim 15, wherein the light probe includes an optical fiber, wherein the optical fiber includes the surface of the first end portion.

17. The system of claim 15, wherein the surface of the first end portion has an angle between 20 and 45 degrees relative to the central longitudinal axis of the light probe.

18. The system of claim 15, wherein the surface of the first end portion is normal to the central longitudinal axis of the light probe.

19. The system of claim 1, wherein the light probe includes an optical fiber bundle with each fiber in the optical fiber bundle including a surface to divert the reflected light.

20. The system of claim 1, wherein a position of the light probe is fixed relative to the light source.

21. The system of claim 1, wherein a central longitudinal axis of the light probe is generally perpendicular to the light beam emitted from the light source.

22. The system of claim 1, wherein a central longitudinal axis of the light probe is generally parallel to the light beam emitted from the light source.

23. The system of claim 1, further comprising a fiber optic cable extending between the second end portion of the light probe and the light sensor.

24. The system of claim 1, wherein the light probe includes a beam splitter and a mirror system to direct the light sample from the beam splitter to the light sensor.

25-75. (canceled)
